(19) United States
(12) Patent Application Publication
(10) Pub. No.: US 2011/0065809 A1
(43) Pub. Date: Mar. 17, 2011

(54) Polymerization of Multifunctional Azides, and Polymers Therefrom

(75) Inventors: Michael Eric Benz, Ramsey, MN (US); Lian Leon Luo, Shoreview, MN (US); Metasebia T. Munie, St. Paul, MN (US)

(73) Assignee: Medtronic, Inc., Minneapolis, MN (US)

(21) Appl. No.: 12/560,240

(22) Filed: Sep. 15, 2009

(51) Int. Cl. A61K 47/30; A61P 43/00; C12P 17/16

(52) U.S. Cl. 514/772.7; 435/118

(57) ABSTRACT

Methods for preparing polymers from multifunctional azides and multifunctional azide-reactants are described in the present disclosure. Exemplary multifunctional azide-reactants include multifunctional alkynes and/or multifunctional α-phosphine esters. In certain embodiments, such polymers can be prepared in vivo. Such polymers can be useful in a wide variety of biomedical applications.

### BACKGROUND

There are numerous biomedical applications for polymers or other materials that can solidify (e.g., gel) after injection in or application to a tissue. However, many materials known in the art that gel after injection in or application to a tissue have drawbacks that limit their usefulness. For example, polymers that solidify upon exposure to ultraviolet (UV) light have been disclosed; however methods of using these polymers can require UV active primers and/or curing agents, multiple steps, and additional curing equipment. For another example, systems that form certain hydrogels have also been disclosed; however such systems can require precise stoichiometry control for multiple reagents, and the reagents, upon mixing, may have limited pot life, which can further require that the reagents be mixed in the operating room.

Thus, there is a continuing need for new polymers and methods of preparing polymers that can solidify after injection or application to a tissue.

### SUMMARY

Bioorthogonal reactions are reactions of materials with each other, wherein each material has limited or substantially no reactivity with functional groups found in vivo. The efficient reaction between an azide and a terminal alkyne, i.e., the most widely studied example of “click” chemistry, is known as a useful example of a bioorthogonal reaction, and has also been reported for use in modifying and/or preparing polymers. However, the preparation of materials that that solidify after injection in, or application to, a tissue using “click” chemistry has not been widely reported.

Described herein are methods for preparing polymers by reacting at least one multifunctional azide with at least one multifunctional azide-reactant (e.g., multifunctional alkynes, multifunctional α-phosphine esters, and combinations thereof). In preferred embodiments, the polymers can be prepared ex vivo or in vivo by introducing at least some of the reactants into a tissue. As used herein, the term “in vivo” refers to a reaction that is within the body of a subject. As used herein, the term “ex vivo” refers to a reaction in tissue (e.g., cells) that has been removed, for example, isolated, from the body of a subject. Tissue that can be removed includes, for example, primary cells (e.g., cells that have recently been removed from a subject and are capable of limited growth or maintenance in tissue culture medium), cultured cells (e.g., cells that are capable of extended growth or maintenance in tissue culture medium), and combinations thereof.

In one aspect, the present invention provides a method of preparing a polymer in a tissue. In one embodiment, the method includes: introducing at least one multifunctional azide into the tissue; introducing at least one multifunctional azide-reactant into the tissue; and allowing the at least one multifunctional azide and the at least one multifunctional azide-reactant to react ex vivo and/or in vivo under conditions effective to form the polymer. In some embodiments, the at least one multifunctional azide-reactant includes at least one multifunctional alkyne (e.g., a terminal alkyne), and the polymer formed is a polytriazole. Optionally, the method further includes providing a source of Cu(I) in the tissue. Such methods can be used, for example, to repair, augment, or replace tissue in need of repair, augmentation, or replacement.

In another aspect, the present invention provides a method of preparing a polymer. In one embodiment, the method includes: combining at least one multifunctional azide and at least one multifunctional cyclic alkyne; and allowing the at least one multifunctional azide and the at least one multifunctional cyclic alkyne to react under conditions effective to form the polymer (e.g., a polytriazole). In preferred embodiments the at least one multifunctional cyclic alkyne is a multifunctional strained cyclic alkyne, and conditions effective for forming the polymer include the substantial absence of added polymerization agent. In some embodiments, the polymer is substantially resistant to hydrolysis under physiological conditions.

In another embodiment of a method of preparing a polymer, the method includes: combining at least one multifunctional azide and at least one multifunctional α-phosphine ester; and allowing the at least one multifunctional azide and the at least one multifunctional α-phosphine ester to react under conditions effective to form the polymer (e.g., a polyamide). In preferred embodiments, conditions effective for forming the polymer include the substantial absence of added polymerization agent. In some embodiments, the polymer is substantially resistant to hydrolysis under physiological conditions.

In another aspect, the present invention provides a medical device including at least one polymer (e.g., a homopolymer or copolymer) including at least two repeat units of the formula (Formula III):

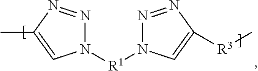

wherein: each R¹ and R³ independently represents an organic group. In certain embodiments the at least one polymer is substantially biostable. Optionally, the medical device further includes at least one biologically active agent that can be at least partially disposed in the at least one polymer.

In another aspect, the present invention provides a composition (e.g., a pharmaceutical composition) including: at least one biologically active agent; and at least one polytriazole including at least two repeat units of the formula (Formula III):

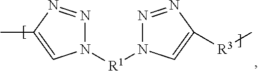

wherein: each R¹ and R³ independently represents an organic group.

In another aspect, the present invention provides a polymer (e.g., a homopolymer or copolymer); compositions including the polymer and at least one biologically active agent (e.g., pharmaceutical compositions); and medical devices including the polymer. The polymer includes at least two repeat units of the formula (Formula VI):

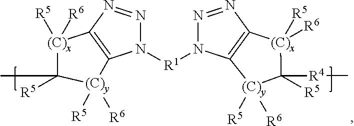

wherein: each R¹ and R⁴ independently represents an organic group; each R⁵ and R⁶ independently represents hydrogen or an organic group; and x and y are each 0 or an integer with the proviso that x+y=2 to 10. In some embodiments the polymer can be applied to a medical device to provide a medical device having the device thereon. In other embodiments the polymer can be applied to a tissue to provide a polymeric coating on the tissue.

In another aspect, the present invention provides a polymer (e.g., a homopolymer or copolymer); compositions including the polymer and at least one biologically active agent (e.g., pharmaceutical compositions); and medical devices including the polymer. The polymer includes at least two repeat units of the formula (Formula VII):

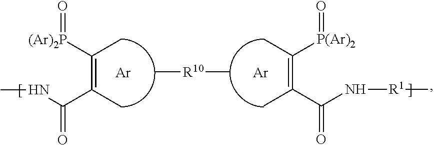

wherein: each R¹ and R¹⁰ independently represents an organic group; the ring structure

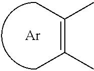

represents an aryl or heteroaryl group; and each Ar independently represents an aryl or a heteroaryl group. In some embodiments the polymer can be applied to a medical device to provide a medical device having the polymer thereon. In other embodiments the polymer can be applied to a tissue to provide a polymeric coating on the tissue.

In another aspect, the present invention provides a method of preparing a medical device. The method includes combining components including: at least one multifunctional azide; and at least one multifunctional azide-reactant, wherein combining includes conditions effective to react the at least one multifunctional azide with the at least one multifunctional azide-reactant to form a polymer. Optionally, the method further includes combining a source of Cu(I).

In another aspect, the present invention provides a method of preparing an active agent delivery system (e.g., a polymeric coating on a medical device). The method includes combining components including: at least one multifunctional azide; at least one multifunctional azide-reactant; and at least one biologically active agent, wherein combining includes conditions effective to react the at least one multifunctional azide with the at least one multifunctional azide-reactant to form a polymer. Optionally, the method further includes combining a source of Cu(I).

In another aspect, the invention provides a method of preparing a medical device having a polymer thereon. The method includes: providing a medical device; and

applying components including at least one multifunctional azide and at least one multifunctional azide-reactant to the device, wherein applying includes conditions effective to react the at least one multifunctional azide with the at least one multifunctional azide-reactant to form a polymer.

In another aspect, the present invention provides a method of preparing a polymeric coating on a tissue. The method includes: providing a tissue; and applying components including at least one multifunctional azide and at least one multifunctional azide-reactant to the tissue, wherein applying includes conditions effective to react the at least one multifunctional azide with the at least one multifunctional azide-reactant to form a polymer.

In another aspect, the present invention provides a method of preparing a medical device having a polymer thereon. The method includes: providing a medical device; and applying at least one polytriazole to at least a portion of the device, wherein the at leak one polytriazole includes at least two repeat units of the formula (Formula III):

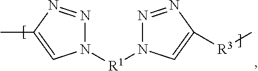

wherein: each R¹ and R³ independently represents an organic group.

In another aspect, the present invention provides a method of preparing a polymeric coating on a tissue. The method includes: providing a tissue; and applying at least one polytriazole to at least a portion of the device, wherein the at least one polytriazole includes at least two repeat units of the formula (Formula III):

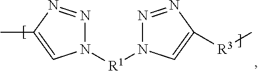

wherein: each R¹ and R³ independently represents an organic group.

The methods and polymers disclosed herein can be used for a wide variety of applications including, for example, delivery of cells, drugs, and/or proteins; repair of intervertebral discs; treatment of vulnerable plaque (gel paving); filling of voids; reinforcement of the esophageal valve; treatment of diabetes through cell implants; repair, augmentation, or replacement of tissue using tissue engineering; repair, augmentation, or replacement of cartilage; and prevention of formation of surgical adhesions. A medical device including a polymer as disclosed herein can be used, for example, to control the release rate of one or more biologically active agents from the device.

### DEFINITIONS

The term “comprises” and variations thereof do not have a limiting meaning where these terms appear in the description and claims.

As used herein, “a,” “an,” “the,” “at least one,” and “one or more” are used interchangeably.

As used herein, the term “or” is generally employed in the sense as including “and/or” unless the context of the usage clearly indicates otherwise.

Also herein, the recitations of numerical ranges by endpoints include all numbers subsumed within that range (e.g., 1 to 5 includes 1, 1.5, 2, 2.75, 3, 3.80, 4, 5, etc.).

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS

Described herein are methods for preparing polymers by reacting at least one multifunctional azide with at least one multifunctional azide-reactant (e.g., multifunctional alkynes, multifunctional α-phosphine esters, and combinations thereof).

As used herein, a “multifunctional azide” refers to a compound that includes two or more azide (—N₃) groups. Exemplary classes of multifunctional azides includes, for example, difunctional azides, trifunctional azides, tetrafunctional azides, pentafunctional azides, hexafunctional azides, heptafunctional azides, octafunctional azides, and combinations thereof.

As used herein, a “multifunctional azide-reactant” refers to a compound that includes two or more groups that can react with an azide group. Exemplary classes of multifunctional azide-reactants include, for example, difunctional azide-reactants, trifunctional azide-reactants, tetrafunctional azide-reactants, pentafunctional azide-reactants, hexafunctional azide-reactants, heptafunctional azide-reactants, octafunctional azide-reactants, and combinations thereof.

When describing the polymers prepared and/or disclosed herein, the term “polymer” is intended to be broadly interpreted to include materials having two or more repeat units, and preferably three or more repeat units. Thus, the term polymer is intended to include materials ranging from oligomers through high molecular weight materials (e.g., materials having a number average molecular weight of at least 1,000 daltons, preferably at least 5,000 daltons, and more preferably at least 10,000 daltons). The term polymer is also intended to encompass both non-crosslinked and crosslinked materials including networks. The term polymer is also intended to include linear, branched, and highly branched materials.

In certain preferred embodiments, the polymers prepared and/or disclosed herein can be a solid or gel. In certain preferred embodiments, the polymers disclosed herein can be hydrophobic or hydrophilic. The polymers prepared and/or disclosed herein can include hydrogels, e.g., polymeric materials that exhibit the ability to swell in water and to retain a significant fraction (e.g., greater than 20 volume %) of water within their structure, but do not dissolve in water), and non-hydrogels. The polymers prepared and/or disclosed herein can include biodegradable materials (e.g., biodegradable materials that do not include ester groups) or biostable materials. As used herein, “biodegradable” and “bioerodible” are used interchangeably and are intended to broadly encompass materials that include, for example, those that tend to break down upon exposure to physiological environments. As used herein, “biostable” is intended to broadly encompass materials that are substantially resistant to hydrolysis under physiological conditions. For example, the function, under physiological conditions, of a device including a material that is substantially resistant to hydrolysis is not substantially affected over the intended functional lifetime of the device.

A single multifunctional azide and a single multifunctional azide-reactant as described herein can be used to prepare a homopolymer. Alternatively, two or more multifunctional azides and/or two or more multifunctional azide-reactants can be used to prepare copolymers. The two or more azides can each be azides having, for example, different structures and/or different functionalities (e.g., difunctional, trifunctional, tetrafunctional, pentafunctional, hexafunctional, heptafunctional, or octafunctional). The two or more azide-reactants can each be, for example, terminal alkynes, cyclic alkynes, strained cyclic alkynes, and/or α-phosphine esters having different structures and/or different functionalities (e.g., difunctional, trifunctional, tetrafunctional, pentafunctional, hexafunctional, heptafunctional, or octafunctional). Further, the two or more azide-reactants can include combinations of, for example, terminal alkynes, cyclic alkynes, strained cyclic alkynes, and/or α-phosphine esters. Copolymers as disclosed herein can be random copolymers, alternating copolymers, block copolymers, graft copolymers, or combinations thereof. Copolymers as disclosed herein can include hard and/or soft segments. For example, mixtures of azides and/or azide-reactants can be combined to prepare random and/or alternating copolymers.

In some embodiments, the at least one multifunctional azide and the at least one multifunctional azide-reactant are each introduced into a tissue and allowed to react ex vivo and/or in vivo, and in certain embodiments in vivo. The at least one multifunctional azide and the at least one multifunctional azide-reactant can be introduced into the tissue substantially simultaneously, or sequentially (e.g., introducing the at least one multifunctional azide occurs prior to introducing the at least one multifunctional azide-reactant, or introducing the at least one multifunctional azide occurs subsequent to introducing the at least one multifunctional azide-reactant).

Conditions effective for the reaction of at least one multifunctional azide with at least one multifunctional azide-reactant can include a polymerization agent (e.g., an added catalyst). A polymerization agent can be used to initiate and/or propagate the polymerization reactions described herein. A wide variety of polymerization agents can be used that are known in the art to catalyze addition polymerizations. Typically, the polymerization agent provides for polymerization through a cationic, an anionic, a free radical, and/or an organometallic pathway. The polymerization agent may be present in catalytic amounts, or alternatively, may be used in stoichiometric amounts with partial or total consumption of the polymerization agent during the polymerization reaction. Alternatively, conditions effective for the reaction of at least one multifunctional azide with at least one multifunctional azide-reactant can be in the substantial absence of added polymerization agent. As used herein, the “substantial absence” of added polymerization agent means that any added polymerization agent increases the rate of polymerization by no more than 10%, preferably by no more than 5%, and more preferably no increase, compared to the rate of polymerization in the complete absence of added polymerization agent.

Typically, the at least one azide and the at least one azide-reactant are introduced in a ratio such that the azide and azide-reactant groups are present in approximately a 1:1 equivalent ratio (e.g., from a 0.95:1 to a 1.05:1 equivalent ratio).

In certain embodiments, the at least one multifunctional azide includes at least one azide of the formula N₃—R¹—N₃, wherein R¹ represents an organic group. For embodiments in which the formed polymer is a hydrogel, R¹ can include, for example, a polyether group (e.g., a poly(ethylene glycol)). An exemplary azide including a polyether group is a diazide of the formula (Formula I):

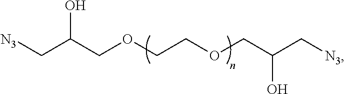

wherein n=2 to 20,000.

As used herein, the term “organic group” is used for the purpose of this invention to mean a hydrocarbon group that is classified as an aliphatic group, cyclic group, or combination of aliphatic and cyclic groups (e.g., alkaryl and aralkyl groups). In the context of the present invention, suitable organic groups for methods and polymers of this invention are those that do not interfere with the polymerization reactions disclosed herein. In the context of the present invention, the term “aliphatic group” means a saturated or unsaturated linear or branched hydrocarbon group. This term is used to encompass alkyl, alkenyl, and alkynyl groups, for example. The term “alkyl group” means a saturated linear or branched monovalent hydrocarbon group including, for example, methyl, ethyl, n-propyl, isopropyl, tert-butyl, amyl, heptyl, and the like. The term “alkenyl group” means an unsaturated, linear or branched monovalent hydrocarbon group with one or more olefinically unsaturated groups (i.e., carbon-carbon double bonds), such as a vinyl group. The term “alkynyl group” means an unsaturated, linear or branched monovalent hydrocarbon group with one or more carbon-carbon triple bonds. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” or “aryl group” means a mono- or polynuclear aromatic hydrocarbon group. The term “heterocyclic group” means a closed ring hydrocarbon in which one or more of the atoms in the ring is an element other than carbon (e.g., nitrogen, oxygen, sulfur, etc.).

As a means of simplifying the discussion and the recitation of certain terminology used throughout this application, the terms “group” and “moiety” are used to differentiate between chemical species that allow for substitution or that may be substituted and those that do not so allow for substitution or may not be so substituted. Thus, when the term “group” is used to describe a chemical substituent, the described chemical material includes the unsubstituted group and that group with nonperoxidic O, N, S, Si, or F atoms, for example, in the chain as well as carbonyl groups or other conventional substituents. Where the term “moiety” is used to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, tert-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ether groups, haloalkyls, nitroalkyls, carboxyalkyls, hydroxyalkyls, sulfoalkyls, etc. On the other hand, the phrase “alkyl moiety” is limited to the inclusion of only pure open chain saturated hydrocarbon alkyl substituents, such as methyl, ethyl, propyl, tert-butyl, and the like.

Thus, for compounds of the formulas as disclosed herein, any of the R substituents that are “organic groups” can include as at least a portion thereof, for example, additional functionality (e.g., azide functionality or azide-reactant functionality). For example, because in the formula N₃—R¹—N₃, R¹ represents an organic group that can include, for example, additional azide groups, the formula N₃—R¹—N₃ represents not only difunctional azides, but can also represent additional multifunctional azides.

Further, for compounds of the formulas as disclosed herein, any of the R substituents that are “organic groups” can include as at least a portion thereof, for example, a group that includes one or more ether groups, ester groups, orthoester groups, ketal groups, carbonate groups, and combinations thereof. Any of the R substituents that are “organic groups” can optionally be polymeric groups including, for example, polyethers, polyesters, poly(orthoesters), polyketals, polycarbonates, and combinations thereof.

Finally, for compounds of the formulas as disclosed herein, any of the R substituents that are “organic groups” can include as at least a portion thereof, for example, an imagable functionality (i.e., a functionality visible in an imaging system, such as, for example, one or more radiopaque functionalities such as iodinated groups, ferromagnetic functionalities, and magnetic susceptible functionalities such as Fe, Cr, Ni, and Gd); a latent reactive functionality (e.g., ethylenic unsaturation and/or oxygen-containing rings suitable for latent crosslinking after polymerization); or combinations thereof.

In one embodiment, the at least one multifunctional azide-reactant can include a multifunctional alkyne, wherein one or more azide groups (—N₃) of the multifunctional azide can react with one or more alkyne groups (—C≡C—) of the multifunctional alkyne to form one or more triazole groups

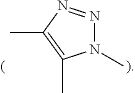

As used herein, a “multifunctional alkyne” refers to a compound that includes two or more alkyne (—C≡C—) groups. The alkyne groups can be terminal alkyne groups (R—C≡C—H), or internal alkyne groups (R—C≡C—R′), which can be either cyclic (e.g., strained or non-strained) or non-cyclic. Exemplary classes of multifunctional alkynes include, for example, di functional alkynes, trifunctional alkynes, tetrafunctional alkynes, pentafunctional alkynes, hexafunctional alkynes, heptafunctional alkynes, octafunctional alkynes, and combinations thereof. The reaction of at least one multifunctional azide with at least one multifunctional alkyne can form a polymer having at least two triazole-containing repeat units (i.e., a polytriazole).

In certain embodiments, the multifunctional alkyne can be a multifunctional terminal alkyne. As used herein, a “multifunctional terminal alkyne” refers to a compound that includes two or more terminal alkyne (—C≡C—H) groups. Exemplary classes of multifunctional terminal alkynes include, for example, difunctional terminal alkynes, trifunctional terminal alkynes, tetrafunctional terminal alkynes, pentafunctional terminal alkynes, hexafunctional terminal alkynes, heptafunctional terminal alkynes, octafunctional terminal alkynes, and combinations thereof. An exemplary class of multifunctional terminal alkynes are alkynes of the formula N—(R²—C≡CH)₃, wherein each R² independently represents an organic group (e.g., an organic moiety), a class of which includes, for example, a trialkyne of the formula (Formula II):

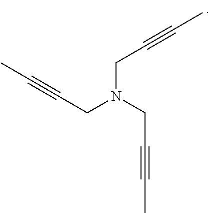

Multifunctional terminal alkynes can be prepared by suitable methods known to one of skill in the art. For example, a polyol can be reacted with a propargyl halide (e.g., propargyl bromide) in the presence of a base.

For embodiments in which the at least one multifunctional azide-reactant includes a multifunctional terminal alkyne, conditions effective for the reaction with the at least one multifunctional azide can sometimes preferably include a polymerization agent (e.g., an added catalyst). Suitable polymerization agents include a source of Cu(I). When Cu(I) is added as a polymerization agent, typically at least 0.1% by weight is added, based on the total weight of the reactants. When Cu(I) is added as a polymerization agent, typically at most 10% by weight is added, based on the total weight of the reactants. In some cases, it may be desirable to generate the Cu(I) catalyst in situ, for example, by reduction of a Cu(II) compound. For example, CuSO₄ can be reduced by sodium ascorbate to generate the desired Cu(I) catalyst in situ.

The reaction of at least one multifunctional azide with at least one multifunctional terminal alkyne can form a polytriazole polymer. Exemplary polymers include those having at least two repeat units of the formula (Formula III):

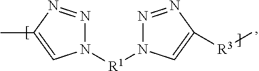

wherein: each R¹ and R³ independently represents an organic group. In certain embodiments, it is preferable that the formed polymer is not a hydrogel. In certain embodiments, the formed polymer is substantially biostable.

In other certain embodiments, the multifunctional alkyne can be a multifunctional cyclic alkyne. As used herein, a “multifunctional cyclic alkyne” refers to a compound that includes two or more cyclic alkyne (—C≡C—) groups. Exemplary classes of multifunctional cyclic alkynes include, for example, difunctional cyclic alkynes, trifunctional cyclic alkynes, tetrafunctional cyclic alkynes, pentafunctional cyclic alkynes, hexafunctional cyclic alkynes, heptafunctional cyclic alkynes, octafunctional cyclic alkynes, and combinations thereof. Preferably, the multifunctional cyclic alkyne is a multifunctional strained cyclic alkyne. As used herein, a “multifunctional strained cyclic alkyne” refers to a compound that includes two or more strained cyclic alkyne (—C≡C—) groups. As used herein, a “strained cyclic alkyne” refers to a cyclic alkyne having at least 8 Kcal/mole strain energy. As used herein, “strain energy” is defined as the difference between the measured heat of formation of the strained cyclic alkyne and the calculated heat of formation of the molecule in a hypothetical strain-free state.

Exemplary multifunctional strained cyclic alkynes include alkynes of the formula (Formula IV):

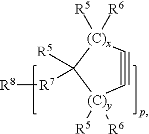

wherein: each R⁵ and R⁶ independently represents hydrogen or an organic group; each R⁷ represents an optional organic linking group; each R⁸ represents an organic group; x and y are each 0 or an integer with the proviso that x+y=2 to 10; and p=2 to 8. In certain embodiments, each R⁵ and R⁶ independently represents hydrogen or an organic moiety; each R⁷ represents an optional organic linking moiety; and each R⁸ represents an organic moiety. Multifunctional strained cyclic alkynes can be prepared by suitable methods known to one of skill in the art. See, for example, Agard et al., J. American Chem. Soc., 126:15046-15047 (2004).

For embodiments in which the at least one multifunctional azide-reactant includes a multifunctional strained cyclic alkyne, conditions effective for the reaction with the at least one multifunctional azide can include the substantial absence of added polymerization agent. The substantial absence of added polymerization agent (e.g., added Cu(I)) can be particularly advantageous in avoiding unintended effects in ex vivo and/or in vivo reaction conditions.

The reaction of at least one multifunctional azide with at least one multifunctional strained cyclic alkyne can form a polytriazole polymer. Exemplary polymers include those having at least two repeat units of the formula (Formula VI):

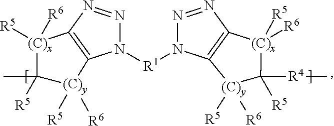

wherein: each R¹ and R⁴ independently represents an organic group; each R⁵ and R⁶ independently represents hydrogen or an organic group; and x and y are each 0 or an integer with the proviso that x+y=2 to 10.

In another embodiment, the at least one multifunctional azide-reactant can include a multifunctional α-phosphine ester, wherein one or more azide groups (—N₃) of the multifunctional azide can react with one or more α-phosphine ester groups

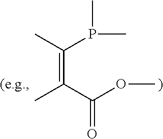

of the multifunctional α-phosphine ester to form one or more amide groups (e.g., —C(O)NH—). As used herein, a “multifunctional α-phosphine ester” refers to a compound that includes two or more α-phosphine ester groups. Exemplary classes of multifunctional α-phosphine esters include, for example, difunctional α-phosphine esters, trifunctional α-phosphine esters, tetrafunctional α-phosphine esters, pentafunctional α-phosphine esters, hexafunctional α-phosphine esters, heptafunctional α-phosphine esters, octafunctional α-phosphine esters, and combinations thereof. The reaction of at least one multifunctional azide with at least one multifunctional α-phosphine ester can form a polymer having at least two amide-containing repeat units (i.e., a polyamide).

Exemplary multifunctional α-phosphine esters include α-phosphine esters of the formula (Formula V):

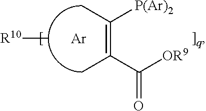

wherein: each R⁹ and R¹⁰ independently represents an organic group; each Ar independently represents an aryl or a heteroaryl group; the ring structure

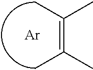

represents an aryl or heteroaryl group in which the indicated vinylic substituents are ortho to one another, and R¹¹ can be at any remaining ring position; and q=2 to 8. In certain embodiments, each R⁹ and R¹⁰ independently represents an organic moiety; the ring structure

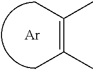

represents an aryl or heteroaryl moiety; and each Ar independently represents an aryl or a heteroaryl moiety. Preferably R⁹ represents methyl. Multifunctional α-phosphine esters can be prepared by suitable methods known to one of skill in the art. See, for example, Saxon et al., Science, 287:2007-2010 (2000).

For embodiments in which the at least one multifunctional azide-reactant includes a multifunctional α-phosphine ester, conditions effective for the reaction with the at least one multifunctional azide can include the substantial absence of added polymerization agent. The substantial absence of added polymerization agent can be particularly advantageous in avoiding unintended effects in ex vivo and/or in vivo reaction conditions.

The reaction of at least one multifunctional azide with at least one multifunctional α-phosphine ester can form a polyamide polymer. Exemplary polymers include those having at least two repeat units of the formula (Formula VII):

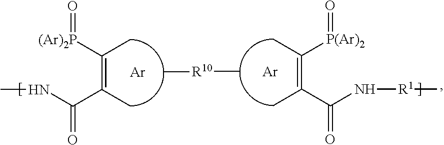

wherein: each R¹ and R¹⁰ independently represents an organic group; the ring structure

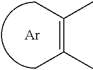

represents an aryl or heteroaryl group; and each Ar independently represents an aryl or a heteroaryl group.

For certain applications, one or more polymers as disclosed herein can be blended with another polymer (e.g., the same or different than the polymers disclosed herein) to provide the desired physical and/or chemical properties. For example, two polytriazole or polyamide polymers having different molecular weights can be blended to optimize the release rate of one or more biologically active agents. For another example, two polytriazole or polyamide polymers having different repeat units can be blended to provide desired physical and/or chemical properties. For another example, a polytriazole polymer and a polyamide polymer can be blended to provide desired physical and/or chemical properties. For even another example, a polytriazole or polyamide polymer can be blended with another polymer that is not a polytriazole or polyamide polymer to provide desired physical and/or chemical properties.

Polymers as disclosed herein can be used in various combinations for various applications. They can be used as tissue-bulking agents in urological applications for bulking the urinary sphincter to prevent stress incontinence or in gastrological applications for bulking of the lower esophageal sphincter to prevent gastroesophageal reflux disease. They can be used for replacements for nucleus pulposis or repair of annulus in intervertebral disc repair procedures. They can be used as tissue adhesives or sealants. They can be used as surgical void fillers, for example, in reconstructive or cosmetic surgery (e.g., for filling a void after tumor removal). They can be used to repair aneurysms, hemorrhagic stroke or other conditions precipitated by failure of a blood vessel. They can be used to prevent surgical adhesions. Polymers as disclosed herein can further be used for applications such as scaffolds or supports for the development and/or growth of cells for applications including, for example, tissue engineering and the fabrication of artificial organs.

Polymers as disclosed herein can be used in injectable compositions. Such injectable compositions could be used as tissue bulking agents (e.g., for the treatment of urinary stress incontinence, for the treatment of gastroesophageal reflux disease, or serving to augment a degenerated intervertebral disc), void fillers (e.g., in cosmetic or reconstructive surgery, such as serving as a replacement for the nucleus pulposis), or as an injectable drug delivery matrix.

In some embodiments, no additives would be needed to form an injectable composition. In some embodiments, one or more polymers can be combined with a solvent such as N-methyl-2-pyrrolidone or dimethylsulfoxide (DMSO), which are fairly biocompatible solvents. The solvent can diffuse away after injection and the polymer can remain in place. Such injectable materials can be applied to a desired site (e.g., a surgical site) using a syringe, catheter, or by hand.

Also, injectable compositions could include crosslinkers (such as diacrylates), plasticizers (such as triethyl citrate), lipids (soybean oil), poly(ethylene glycol) (including those with the ends blocked with methyls or similar groups), silicone oil, partially or fully fluorinated hydrocarbons, N-methyl-2-pyrrolidone, or mixtures thereof.

In certain embodiments, polymers as disclosed herein can be used, for example, to repair, augment, or replace tissue in need of repair, augmentation, or replacement. In one embodiment, at least one multifunctional azide and at least one multifunctional azide-reactant can be introduced proximate a tissue in need of repair, augmentation, or replacement and the at least one multifunctional azide and the at least one multifunctional azide-reactant allowed to react ex vivo and/or in vivo under conditions effective to form a polymer.

Polymers as disclosed herein can be used in combination with a variety of particulate materials. For example, they can be used with moisture curing ceramic materials (e.g., tricalcium phosphate) for vertebroplasty cements, bone void filling (due to disease such as cancer or due to fracture). They can be used in combination with inorganic materials such as hydroxylapatite to form pastes for use in bone healing, sealing, filling, repair, augmentation, and replacement. They can be used as or in combination with polymer microspheres that can be reservoirs for one or more biologically active agents such as a protein, DNA plasmid, RNA plasmid, antisense agent, etc. Alternatively, polymers as disclosed herein can be used in combination with other materials to form a composite (e.g., a polymer having an additive therein). In addition to one or more polymers as disclosed herein, composites can include a wide variety of additives, and particularly particulate additives, such as, for example, fillers (e.g., including particulate, fiber, and/or platelet material), other polymers (e.g., polymer particulate materials such as polytetrafluoroethylene can result in higher modulus composites), imaging particulate materials (e.g., barium sulfate for visualizing material placement using, for example, fluoroscopy), biologically derived materials (e.g., bone particles, cartilage, demineralized bone matrix, platelet gel, and combinations thereof), and combinations thereof. Additives can be dissolved, suspended, and/or dispersed within the composite. For particulate additives, the additive is typically dispersed within the composite.

Polymers as disclosed herein can be combined with fibers, woven or nonwoven fabric for reconstructive surgery, such as the in situ formation of a bone plate or a bone prosthesis.

In certain embodiments, one or more polymers as disclosed herein can be shaped to form a medical device. The one or more polymers can be shaped by methods known in the art including compression molding, injection molding, casting, extruding, milling, blow molding, or combinations thereof. As used herein, a “medical device” includes devices that have surfaces that contact tissue, bone, blood, or other bodily fluids in the course of their operation, which fluids are subsequently used in patients. This can include, for example, extracorporeal devices for use in surgery such as blood oxygenators, blood pumps, blood sensors, tubing used to carry blood, and the like which contact blood which is then returned to the patient. This can also include endoprostheses implanted in blood contact in a human or animal body such as vascular grafts, stents, pacemaker leads, heart valves, and the like, that are implanted in blood vessels or in the heart. This can also include devices for temporary intravascular use such as catheters, guide wires, and the like which are placed into the blood vessels or the heart for purposes of monitoring or repair. A medical device can also be fabricated by reacting at least one azide and at least one azide-reactant in a suitable mold under conditions effective to form a polymer. Polymers as disclosed herein can also be coated onto a substrate if desired. A coating mixture of the polymer can be prepared using a wide variety of solvents including, but not limited to, water, ether, ethyl acetate, alcohols, toluene, chloroform, tetrahydrofuran, perfluorinated solvents, and combinations thereof. Preferred solvents include water, ether, ethyl acetate and low molecular weight alcohols (less than eight carbons). The coating mixture can be applied to an appropriate substrate such as uncoated or polymer coated medical wires, catheters, stents, prostheses, penile inserts, and the like, by conventional coating application methods. Such methods include, but are not limited to, dipping, spraying, wiping, painting, solvent swelling, and the like. After applying the coating solution to a substrate, the solvent is preferably allowed to evaporate from the coated substrate.

The materials of a suitable substrate include, but are not limited to, polymers, metal, glass, ceramics, composites, and multilayer laminates of these materials. The coating may be applied to metal substrates such as the stainless steel used for guide wires, stents, catheters and other devices. Organic substrates that may be coated with the polymers of this invention include, but are not limited to, polyether-polyamide block copolymers, polyethylene terephthalate, polyetherurethane, polyesterurethane, other polyurethanes, silicone, natural rubber, rubber latex, synthetic rubbers, polyester-polyether copolymers, polycarbonates, and other organic materials.

Additives that can be combined with a polymer as disclosed herein to form a composition include, but are not limited to, wetting agents for improving wettability to hydrophobic surfaces, viscosity and flow control agents to adjust the viscosity and thixotropy of the mixture to a desired level, tackifiers, adhesion promoters, antioxidants to improve oxidative stability of the coatings, dyes or pigments to impart color or radiopacity, and air release agents or defoamers, cure catalysts, cure accelerants, plasticizers, solvents, stabilizers (cure inhibitors, pot-life extenders), and adhesion promoters.

Additionally, if the one or more multifunctional azides and the one or more multifunctional azide-reactants are selected so that they also do not react with a therapeutic agent of interest (that is, they are also pharmaorthogonal), they can be used to create matrices to deliver drugs, proteins, DNA, or other therapeutic agents.

Of particular interest for medical and pharmaceutical applications are compositions that include one or more polymers as disclosed herein and at least one biologically active agent. As used herein, a “biologically active agent” is intended to be broadly interpreted as any agent capable of eliciting a response in a biological system such as, for example, living cell(s), tissue(s), organ(s), and being(s). Biologically active agents can include natural and/or synthetic agents. Thus, a biologically active agent is intended to be inclusive of any substance intended for use in the diagnosis, cure, mitigation, treatment, or prevention of disease or in the enhancement of desirable physical or mental development and conditions in a subject.

The term “subject” as used herein is taken to include humans, sheep, horses, cattle, pigs, dogs, cats, rats, mice, birds, reptiles, fish, insects, arachnids, protists (e.g., protozoa), and prokaryotic bacteria. Preferably, the subject is a human or other mammal.

A preferred class of biologically active agents includes drugs. As used herein, the term “drug” means any therapeutic agent. Suitable drugs include inorganic and organic drugs, without limitation, and include drugs that act on the peripheral nerves, adrenergic receptors, cholinergic receptors, nervous system, skeletal muscles, cardiovascular system, smooth muscles, blood circulatory system, synaptic sites, neuro-effector junctional sites, endocrine system, hormone systems, immunological system, reproductive system, skeletal system, autocoid systems, alimentary and excretory systems (including urological systems), histamine systems, and the like. Such conditions, as well as others, can be advantageously treated using compositions as disclosed herein.

Suitable drugs include, for example, polypeptides (which is used herein to encompass a polymer of L- or D-amino acids of any length including peptides, oligopeptides, proteins, enzymes, hormones, etc.), polynucleotides (which is used herein to encompass a polymer of nucleic acids of any length including oligonucleotides, single- and double-stranded DNA, single- and double-stranded RNA, DNA/RNA chimeras, etc.), saccharides (e.g., mono-, di-, poly-saccharides, and mucopolysaccharides), vitamins, viral agents, and other living material, radionuclides, and the like. Examples include antithrombogenic and anticoagulant agents such as heparin, coumadin, protamine, and hirudin; antimicrobial agents such as antibiotics; antineoplastic agents and anti-proliferative agents such as etoposide, podophylotoxin; antiplatelet agents including aspirin and dipyridamole; antimitotics (cytotoxic agents) and antimetabolites such as methotrexate, colchicine, azathioprine, vincristine, vinblastine, fluorouracil, adriamycin, and mutamycinnucleic acids; antidiabetic such as rosiglitazone maleate; and anti-inflammatory agents. Anti-inflammatory agents for use in the present invention include glucocorticoids, their salts, and derivatives thereof, such as cortisol, cortisone, fludrocortisone, Prednisone, Prednisolone, 6α-methylprednisolone, triamcinolone, betamethasone, dexamethasone, beclomethasone, aclomethasone, amcinonide, clebethasol and clocortolone.

Illustrative classes of drugs include, for example, Plasmid DNA, genes, antisense oligonucleotides and other antisense agents, peptides, proteins, protein analogs, siRNA, shRNA, miRNA, ribozymes, DNAzymes and other DNA based agents, viral and non-viral vectors, lyposomes, cells, stem cells, antineoplastic agents, antiproliferative agents, antithrombogenic agents, anticoagulant agents, antiplatelet agents, antibiotics, anti-inflammatory agents, antimitotic agents, immunosuppressants, growth factors, cytokines, hormones, and combinations thereof.

Suitable drugs can have a variety of uses including, but are not limited to, anticonvulsants, analgesics, antiparkinsons, antiinflammatories (e.g., ibuprofen, fenbufen, cortisone, and the like), calcium antagonists, anesthetics (e.g., benoxinate, benzocaine, procaine, and the like), antibiotics (e.g., ciprofloxacin, norfloxacin, clofoctol, and the like), antimalarials, antiparasitics, antihypertensives, antihistamines, antipyretics, alpha-adrenergic agonists, alpha-blockers, biocides, bactericides, bronchial dilators, beta-adrenergic blocking drugs, contraceptives, cardiovascular drugs, calcium channel inhibitors, depressants, diagnostics, diuretics, electrolytes, enzymes, hypnotics, hormones, hypoglycemics, hyperglycemics, muscle contractants, muscle relaxants, neoplastics, glycoproteins, nucleoproteins, lipoproteins, ophthalmics, psychic energizers, sedatives, steroids sympathomimetics, parasympathomimetics, tranquilizers, urinary tract drugs, vaccines, vaginal drugs, vitamins, collagen, hyaluronic acid, nonsteroidal anti-inflammatory drugs, angiotensin converting enzymes, polynucleotides, polypeptides, polysaccharides, and the like.

Certain embodiments include a drug selected from the group consisting of indomethacin, sulindac, diclofenal, etodolac, meclofenate, mefenamic acid, nambunetone, piroxicam, phenylgutazone, meloxicam, dexamethoasone, betamethasone, dipropionate, diflorsasone diacetate, clobetasol propionate, galobetasol propionate, amcinomide, beclomethasone dipropionate, fluocinomide, betamethasone valerate, triamcinolone acetonide, penicillamine, hydroxychloroquine, sulfasalazine, azathioprine, minocycline, cyclophosphamide, methotrexate, cyclosporine, leflunomide, etanercept, infliximab, ascomycin, beta-estradiol, rosiglitazone, troglitazone, pioglitazone, S-nitrosoglutathione, gliotoxin G, panepoxydone, cycloepoxydon tepoxalin, curcumin, a proteasome inhibitor (e.g., bortezomib, dipeptide boronic acid, lactacystin, bisphosphonate, zolendronate, epoxomicin), antisense c-myc, celocoxib, valdecoxib, and combinations thereof. Certain embodiments include a drug selected from the group consisting of podophyllotoxin, mycophenolic acid, teniposide, etoposide, trans-retinoic acids, 9-cis retinoic acid, 13-cis retinoic acid, rapamycin, a rapalog (e.g., Everolimus, ABT-578), camptothecin, irinotecan, topotecan, tacromilus, mithramycin, mitobronitol, thiotepa, treosulfan, estramusting, chlormethine, carmustine, lomustine, busultan, mephalan, chlorambucil, ifosfamide, cyclophosphamide, doxorubicin, epirubicin, aclarubicin, daunorubicin, mitosanthrone, bleomycin, cepecitabine, cytarabine, fludarabine, cladribine, gemtabine, 5-fluorouracil, mercaptopurine, tioguanine, vinblastine, vincristine, vindesine, vinorelbine, amsacrine, bexarotene, crisantaspase, decarbasine, hydrosycarbamide, pentostatin, carboplatin, cisplatin, oxiplatin, procarbazine, paclitaxel, docetaxel, epothilone A, epothilone B, epothilone D, baxiliximab, daclizumab, interferon alpha, interferon beta, maytansine, and combinations thereof.

Certain embodiments include a drug selected from the group consisting of salicylic acid, fenbufen, cortisone, ibuprofen, diflunisal, sulindac, difluprednate, prednisone, medrysone, acematacin, indomethacin, meloxicam, camptothecin, benoxinate, benzocaine, procaine, ciprofloxacin, norfloxacin, clofoctol, dexamethasone, fluocinolone, ketorolac, pentoxifylline, rapamycin, ABT-578, gabapentin, baclofen, sulfasalazine, bupivacaine, sulindac, clonidine, etanercept, pegsunercept, and combinations thereof.

Compositions including at least one biologically active agent and at least one polymer as disclosed herein and can be prepared by suitable methods known in the art. For example, such compositions can be prepared by solution processing, milling, extruding, and/or reacting components including multifunctional azides and multifunctional azide-reactants in the presence of at least one biologically active agent.

Compositions including polymers as disclosed herein (e.g., with or without a biologically active agent) can further include additional components. Examples of such additional components include fillers, dyes, pigments, inhibitors, accelerators, viscosity modifiers, tackifiers, adhesion promoters, wetting agents, buffering agents, stabilizers, biologically active agents, polymeric materials, excipients, and combinations thereof.

Medical devices that include one or more polymers as disclosed herein and one or more biologically active agents can have a wide variety of uses. In such devices, the one or more biologically active agents are preferably disposed in the one or more polymers. As used herein, the term “disposed” is intended to be broadly interpreted as inclusive of dispersed, dissolved, suspended, or otherwise contained at least partially therein or thereon.

For example, such devices can be used to deliver one or more biologically active agents to a tissue by positioning at least a portion of the device including the one or more polymers proximate the tissue and allowing the one or more polymers to deliver the one or more biologically active agents disposed therein. For another example, such devices can be used to control the release rate of one or more biologically active agents from a medical device by disposing the one or more biologically active agents in at least one of the one or more polymers.

The present invention is illustrated by the following examples. It is to be understood that the particular examples, materials, amounts, and procedures are to be interpreted broadly in accordance with the scope and spirit of the invention as set forth herein.

### EXAMPLES

Unless otherwise noted, all solvents and reagents were or can be obtained from Sigma-Aldrich Corp., St. Louis, Mo.

### Preparatory Example 1

### Preparation of a Diazide of the Formula (Formula I)

Polyethylene glycol diglycidyl ether, Mₙ˜526 (7.89 g; 0.015 mole) was added to a solution of sodium azide (NaN₃; 9.75 g; 0.15 mole) in 60 milliliters (ml) water. The pH of the solution was measured to be 12.2. The solution was allowed to stir overnight. A small sample of the solution was taken out and concentrated under full vacuum. The product was then dissolved in D₂O, and ¹H and ¹³C NMR spectra were acquired. The ¹³C NMR spectrum was consistent with the formation of an azido alcohol of Formula I. Before extraction, 20 ml of a saturated aqueous NaCl solution was put into the reaction mixture to reduce the solubility of the azido alcohol in the aqueous phase. Diethyl ether (100 ml) was put into a separatory funnel and the reaction mixture was poured into the ether, producing two layers. Both layers were collected, and both the aqueous and the ether layers were each re-extracted two more times with ether. The collected ether layers were then combined and dried under sodium sulfate (approximately 10 g). After 30 minutes, the ether layer was then poured into a round bottomed flask and concentrated under full vacuum to remove the ether, giving the azido alcohol in 39% yield.

### Preparatory Example 2

### Preparation of an Azide-Terminated Polyethylene Glycol

Polyethylene glycol, Mₙ=600 (PEG 600) was dried using a rotary evaporator at a pressure of 20 torr (2.7 kilopascals) and heating at 80° C. in an oil bath. The dried PEG 600 (20 g; 0.033 moles) was then dissolved in 150 ml of anhydrous tetrahydrofuran (THF), and triethylamine (7.0 g; 9.7 ml; 0.0693 moles) was added. Methanesulfonyl chloride (MsCl; 7.94 g; 0.0693 moles) was separately dissolved in anhydrous THF (20 ml). The PEG 600 solution was cooled to 0° C. and the MsCl solution was added drop wise to it to give a 2.1:1 molar ratio of the MsCl to PEG 600. A white solid (triethylamine hydrochloride) precipitated from solution. The reaction mixture was warmed to room temperature and stirred overnight under a nitrogen atmosphere. The precipitate was then filtered out of solution, and the filtrate concentrated under full vacuum at 50° C. A sample of the dried product was dissolved in d₈-THF, and ¹H and ¹³C NMR spectra were acquired, which were consistent with the mesylated-PEG 600 (25.34 g; 0.0335 moles). The mesylated-PEG 600 was dissolved in acetone (50 ml), sodium azide (5.45 g; 0.0838 moles; 2.5 equivalents of azide to mesylate) was added, and the reaction mixture was stirred for 2 days. A sample of the solution was then concentrated under reduced pressure to remove the solvent, the product was dissolved in d₆-acetone, and ¹H and ¹³C NMR spectra were acquired. The spectra showed that some mesylated-PEG 600 was still present. Additional sodium azide (2 g) was added and the solution was warmed at low heat and thickened overnight. A sample of the reaction mixture was then concentrated under reduced pressure to remove the solvent, the product was dissolved in 4-acetone, and a ¹³C NMR spectrum was acquired. The spectrum showed that the mesylated-PEG 600 had disappeared.

### Preparatory Example 3

### Preparation of a Propargyl-Terminated Polyethylene Glycol

Polyethylene glycol, Mₙ=600 (PEG 600) was dried using a rotary evaporator at a pressure of 20 torr (2.7 kilopascals) and heating at 80° C. in an oil bath. The dried PEG 600 (10 g; 0.017 moles) was then dissolved in 100 ml of anhydrous tetrahydrofuran (THF), and 0.78 g of Na metal (0.034 moles) was added under a continuous nitrogen purge. The reaction mixture was stirred over the weekend to allow all the Na metal to react, then cooled to 0° C. in an ice bath. In a separate vessel, propargyl bromide (5.06 g, 0.043 moles) was dissolved in 10 ml of anhydrous THF, and the resulting solution was added dropwise to the PEG reaction mixture. The color of the reaction mixture was initially brown, but it changed to green after 3 days. A small sample was taken from the reaction mixture and concentrated under full vacuum and ambient temperature. The dried product was dissolved in CDCl₃ and a ¹³C NMR spectrum was obtained, which indicated that a small amount of unreacted PEG remained. Additional propargyl bromide (0.25 equivalents to PEG) was added, but unreacted PEG still remained. The whole solution was then concentrated under reduced pressure to give the product, which was a thick green liquid.

### Example 1

Tripropargyl amine (0.33 g; 0.0025 mole) was added to the azido alcohol prepared in Preparatory Example 1 (2.31 g; 0.0038 moles) in a round bottomed flask (2:3 molar ratio of the tripropargyl amine to the azido alcohol). CuSO₄ (0.139 g; 5% by weight) and sodium ascorbate (0.293 g; 10% by weight) were dissolved in 15 ml of water to generate a Cu(I) catalyst in situ. The aqueous Cu(I) catalyst solution was then added to the flask containing the azido alcohol and the tripropargyl amine, and the reaction mixture was stirred until becoming thick overnight. Additional water (30 ml) was added to the flask, and the product was separated by centrifuging the reaction mixture. The resulting polymer was then dried at ambient temperature under full vacuum.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A method of preparing a polymer in a tissue, the method comprising:
introducing at least one multifunctional azide into the tissue;
introducing at least one multifunctional azide-reactant into the tissue; and
allowing the at least one multifunctional azide and the at least one multifunctional azide-reactant to react ex vivo and/or in vivo under conditions effective to form the polymer.

2-7. (canceled)

8. The method of claim 1 wherein the at least one multifunctional azide comprises at least one azide of the formula N₃—R¹—N₃, wherein R¹ represents an organic group.

9. The method of claim 8 wherein R¹ comprises a polyether.

10. The method of claim 9 wherein the polyether is a poly(ethylene glycol).

11. The method of claim 10 wherein the at least one azide is a diazide of the formula (Formula I): 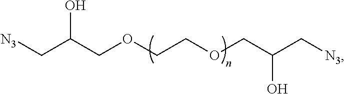
wherein n=2 to 20,000.

12. The method of claim 1 wherein the at least one multifunctional azide-reactant comprises at least one multifunctional alkyne.

13-18. (canceled)

19. The method of claim 12 wherein the at least one multifunctional alkyne comprises a multifunctional terminal alkyne.

20. (canceled)

21. (canceled)

22. The method of claim 19 wherein the multifunctional terminal alkyne is an alkyne of the formula N—(R²—C≡CH)₃, wherein each R² independently represents an organic group.

23. The method of claim 22 wherein each R² independently represents an organic moiety.

24. The method of claim 23 wherein the multifunctional terminal alkyne is a trialkyne of the formula (Formula II):

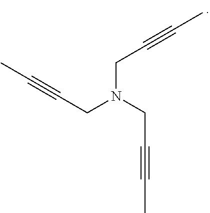

25-54. (canceled)

55. A medical device comprising at least one polymer comprising at least two repeat units of the formula (Formula III): 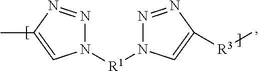
wherein:
each R¹ and R³ independently represents an organic group.

56. The medical device of claim 55 wherein the at least one polymer is not a hydrogel.

57. The medical device of claim 55 wherein the at least one polymer is substantially biostable.

58. The medical device of claim 55 wherein the at least one polymer is biodegradable.

59. The medical device of claim 58 wherein the at least one polymer does not comprise ester groups.

60. The medical device of claim 55 wherein the at least one polymer is a copolymer.

61. The medical device of claim 55 wherein the device further comprises at least one biologically active agent.

62. The medical device of claim 61 wherein the at least one biologically active agent is at least partially disposed in the at least one polymer.

63. A composition comprising:
at least one biologically active agent; and
at least one polytriazole comprising at least two repeat units of the formula (Formula III): 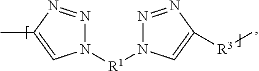
wherein:
each R¹ and R³ independently represents an organic group.

64. The composition of claim 63 wherein the composition is a pharmaceutical composition.

65-108. (canceled)
